(19) United States
(12) Patent Application Publication
(10) Pub. No.: US 2022/0151601 A1
(43) Pub. Date: May 19, 2022

(54) MULTIPURPOSE SURGICAL RETRACTOR AND METHODS OF USE

(71) Applicant: NEUROENTERPRISES LLC, Chicago, IL (US)

(72) Inventors: Giuseppe Gallo, Naperville, IL (US); Ali Hemmati, Chicago, IL (US); Sean Salehi, Chicago, IL (US)

(21) Appl. No.: 17/583,359

(22) Filed: Jan. 25, 2022

Related U.S. Application Data
(63) Continuation of application No. PCT/US2020/018969, filed on Feb. 20, 2020.
(60) Provisional application No. 62/922,103, filed on Jul. 25, 2019.

(51) Int. Cl. A61B 17/02

(52) U.S. Cl. CPC A61B 17/0206; A61B 2017/00946

(57) ABSTRACT

Provided herein are systems, methods and apparatus for a Multi-purpose Surgical Retractor.

### CROSS-REFERENCE TO RELATED APPLICATIONS

The present application claims priority to PCT application serial no. PCT/US2020/018969, filed on Feb. 20, 2020, which claims priority to US provisional application Ser. No. 62/922,103, filed on Jul. 25, 2019, each herein incorporated by reference in their entireties.

### BACKGROUND

The invention generally relates to surgical tools. Tissue retraction is a very common in open surgical procedures. The common retractors used in surgeries today have either fixed or limited angle of adjustment. The present invention attempts to solve these problems & others.

### SUMMARY OF THE INVENTION

Provided herein are systems, methods and apparatus for a Multi-purpose Surgical Retractor. The methods, systems, and apparatuses are set forth in part in the description which follows, and in part will be obvious from the description, or can be learned by practice of the methods, apparatuses, and systems. The advantages of the methods, apparatuses, and systems will be realized and attained by means of the elements and combinations particularly pointed out in the appended claims. It is to be understood that both the foregoing general description and the following detailed description are exemplary and explanatory only and are not restrictive of the methods, apparatuses, and systems, as claimed.

### BRIEF DESCRIPTION OF THE DRAWINGS

In the accompanying figures, like elements are identified by like reference numerals among the several preferred embodiments of the present invention. FIG. 1A is a perspective view of the Multipurpose Retractor in the closed position, according to one embodiment; FIG. 1B is a perspective view of the Multipurpose Retractor in the bent configuration and the closed position, according to one embodiment; FIG. 1C is a perspective view of the Multipurpose Retractor in the open position and the bent configuration; FIG. 1D is an exploded view of the Multipurpose Retractor, according to one embodiment; and FIG. 1E is a top view of the plurality of links rotatable about an axis with respect to an adjacent link.

FIG. 2A is a side view of the handle and the trigger mechanism in the closed position; FIG. 2B is a top perspective view of the handle and the trigger mechanism in the open position.

FIG. 3A is an exploded view of the first arm and the second arm coupled with the plurality of links and pins; FIG. 3B is a cross-sectional view of the first arm coupled with the plurality of links and plurality of pins.

FIG. 4A is a perspective exploded view of the plurality of links coupled with the first mating junction and the second mating junction with the first retractor and the second retractor, and FIG. 4B is a side view of the plurality of links and the distal claw, according to one embodiment.

FIG. 5A is perspective view of the multipurpose retractor in the open position and bent configuration with the second adjustable mechanism bent about 180 degrees from the original longitudinal position; and FIG. 5B is a perspective view of the multipurpose retractor in the open position and bent configuration with the second adjustable mechanism bent opposite from the first adjustable mechanism.

FIG. 6A is a perspective view of the multipurpose retractor in the closed position with an alternative first retractor as a hook embodiment; FIG. 6B is a perspective view of the multipurpose retractor in the closed position with an alternative first retractor and the second retractor as curved hooks; FIG. 6C is a perspective view of the multipurpose retractor in the closed position with an alternative first retractor and the second retractor as curved plates; FIG. 6D is a perspective view of the multipurpose retractor in the closed position with an alternative first retractor and the second retractor as wide curved plates.

FIG. 7A is a side view of the link; and FIG. 7B is a top view of the link.

FIG. 8A is a side view of an alternative adjustable mechanism as a plurality of cylinders; FIG. 8B is a side view of an alternative adjustable mechanism as a metal cable; FIG. 8C is a side view of an alternative adjustable mechanism as a plurality of interconnected spheres; and FIG. 8D is a side view of an alternative adjustable mechanism as a braided polymer.

### DETAILED DESCRIPTION OF THE INVENTION

The foregoing and other features and advantages of the invention are apparent from the following detailed description of exemplary embodiments, read in conjunction with the accompanying drawings. The detailed description and drawings are merely illustrative of the invention rather than limiting, the scope of the invention being defined by the appended claims and equivalents thereof.

Embodiments of the invention will now be described with reference to the Figures, wherein like numerals reflect like elements throughout. The terminology used in the description presented herein is not intended to be interpreted in any limited or restrictive way, simply because it is being utilized in conjunction with detailed description of certain specific embodiments of the invention. Furthermore, embodiments of the invention may include several novel features, no single one of which is solely responsible for its desirable attributes or which is essential to practicing the invention described herein. The words proximal and distal are applied herein to denote specific ends of components of the instrument described herein. A proximal end refers to the end of an instrument nearer to an operator of the instrument when the instrument is being used. A distal end refers to the end of a component further from the operator and extending towards the surgical area of a patient and/or the implant.

The use of the terms “a” and “an” and “the” and similar referents in the context of describing the invention are to be construed to cover both the singular and the plural, unless otherwise indicated herein or clearly contradicted by context. It will be further understood that the terms “comprises,” “comprising,” “includes,” and/or “including,” when used herein, specify the presence of stated features, integers, steps, operations, elements, and/or components, but do not preclude the presence or addition of one or more other features, integers, steps, operations, elements, components, and/or groups thereof.

Recitation of ranges of values herein are merely intended to serve as a shorthand method of referring individually to each separate value falling within the range, unless otherwise indicated herein, and each separate value is incorporated into the specification as if it were individually recited herein. The word “about,” when accompanying a numerical value, is to be construed as indicating a deviation of up to and inclusive of 10% from the stated numerical value. The use of any and all examples, or exemplary language (“e.g.” or “such as”) provided herein, is intended merely to better illuminate the invention and does not pose a limitation on the scope of the invention unless otherwise claimed. No language in the specification should be construed as indicating any nonclaimed element as essential to the practice of the invention.

References to “one embodiment,” “an embodiment,” “example embodiment,” “various embodiments,” etc., may indicate that the embodiment(s) of the invention so described may include a particular feature, structure, or characteristic, but not every embodiment necessarily includes the particular feature, structure, or characteristic. Further, repeated use of the phrase “in one embodiment,” or “in an exemplary embodiment,” do not necessarily refer to the same embodiment, although they may.

As used herein the term “method” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical and medical arts. Unless otherwise expressly stated, it is in no way intended that any method or aspect set forth herein be construed as requiring that its steps be performed in a specific order. Accordingly, where a method claim does not specifically state in the claims or descriptions that the steps are to be limited to a specific order, it is no way intended that an order be inferred, in any respect. This holds for any possible non-express basis for interpretation, including matters of logic with respect to arrangement of steps or operational flow, plain meaning derived from grammatical organization or punctuation, or the number or type of aspects described in the specification.

In one embodiment, the Multipurpose Retractor employs a multi-adjustable mechanism in the midsection of the retractor allowing for quick, easy and versatile retraction. Such a design obviates the need for carrying multiple styles or sizes of the reactor needed to suit patient's anatomy or size. The Multipurpose Retractor modular nature also allows for the same handle to be used with different retractors or blades.

In one embodiment, the Multipurpose Retractor is used in surgeries, including but not limited, spina surgery, general orthopedics, trauma surgery, general surgery, plastic surgery, spinal orthopedics, Ear Nose and Throat (ENT) procedures, neurosurgical procedures, cardiac procedures, veterinary surgery, and any surgical other procedures. Multipurpose Retractor replaces the need for different retractors which can be costly and time effective during surgical procedure. Multipurpose Retractor further allows intra-operative adjustability to fit the retractor to the patient's anatomy, such as adjustability to the depth of field, which allows an unencumbered surgical field and better visualization. The Multipurpose Retractor also allows the remainder of the handle outside the surgical corridor to rest flushed with the rest of skin so the surgeon's hands can get closer to the operated anatomy and not encumbered but the handle pointing in the air like existing retractors.

As shown in FIG. 1A, the Multipurpose Retractor 100 comprises a first handle 110 and a second handle 112, wherein the first handle 110 includes a first arm 120 and the second handle 112 includes a second arm 130 extending to a distal end. A first adjustable mechanism 140 extends from the first arm 120 and a second adjustable mechanism extends from the second arm 130, wherein the first adjustable mechanism and the second adjustable mechanism pivot about a longitudinal axis 102 by angle A1 from an unbent configuration, as shown in FIG. 1A of the Multipurpose Retractor 100 to a bent configuration, as shown in FIG. 1B, and according to one embodiment. In other embodiment, the Multipurpose Retractor includes other bent configurations. The longitudinal axis 102 is generally shown as the x-axis, while the vertical axis is the y-axis, and the lateral axis is the z-axis. In alternative embodiments, the first adjustable mechanism and the second adjustable mechanism pivot about the vertical axis or the lateral axis. A first retractor 160 extends from the first adjustable mechanism 140 and a second retractor 170 extending from the second adjustable mechanism 150, wherein the first retractor 160 and second retractor 170 are operable to retract tissue when the Multipurpose Retractor moves to the open position in the bent configuration, as shown in FIG. 1C. The first arm 120 and the second arm 130 are operable in a closed position and an open position through a pivot point 180 disposed between the first arm and a first handle and the pivot point disposed between the second arm and a second handle.

In one embodiment, the first retractor 160 and the second retractor 170 includes a first distal claw 168 and the second retractor includes a second distal claw 178, wherein the first distal claw 168 and the second distal claw 178 are operable to retract tissue when operable from the closed position to the open position. In alternative embodiments, the first retractor 160 and the second retractor 170 are selected from the group of hooks, blades, rakes, and combinations thereof. The first distal claw 168 includes a length L1 and the second distal claw 178 includes a length L2, wherein L1 and L2 are equal and a length to retract tissue, according to one embodiment. In other embodiments, length L1 and L2 are unequal to accommodate a particular tissue or surgical procedure. In one embodiment, the first distal claw 168 and the second distal claw 178 are a straight rake.

In one embodiment, the first handle 110 includes a first gripping portion 114 on the proximal end and the second handle 112 includes a second gripping portion 116 on the proximal end; and the first gripping portion and the second gripping portion assist in pivoting the first arm and the second arm about the pivot point from the closed position to the open position. In one embodiment, the first gripping portion 114 and the second gripping portion 116 are in a circular configuration. In alternative embodiments, the first gripping portion 114 and the second gripping portion 116 are in a polygonal, rectangular, elliptical, or triangular configurations. The first handle 110 and the second handle 112 operate with different adjustable mechanisms by way of the different attachment points, either by pins, locks, gears, and the like.

In another embodiment, the first adjustable mechanism 140 and the second adjustable mechanism 150 pivot about the longitudinal axis and the vertical axis. In another embodiment, the first adjustable mechanism and the second adjustable mechanism pivot about the longitudinal axis, the vertical axis, and the lateral axis. In another embodiment, the first adjustable mechanism and the second adjustable mechanism pivot about the vertical axis and the lateral axis. The determining factor of how the first adjustable mechanism and the second adjustable mechanism pivot about the longitudinal axis, the lateral axis, or the vertical axis is the type mechanism the first adjustable mechanism and the second adjustable mechanism. For the interconnected links, the first adjustable mechanism and the second adjustable mechanism connection to the first arm 120 and the second arm 130 may rotate about 45 degrees to adjust the pivot axis to the longitudinal axis, the vertical axis, or the lateral axis. The first adjustable mechanism and the second adjustable mechanism include mechanisms selected from the group consisting of: interconnected links, chains, linked spheres, braided polymers, cylinders, memory metals, fluid hydraulics, and metal cables. In one embodiment, adjustability can be accomplished via the use of malleable materials such as shape memory alloys and shape memory polymers. In one embodiment, the interconnected links are fixed in position and prevented from further rotation when in the open position by methods selected from the group consisting of frictional pivots, memory metal, fluid hydraulics, or temperature sensitive materials. In one embodiment, each link includes a pivot movement of about 90 degrees in each direction whether the surgical retractor is in the open position or the closed position. When the surgical retractor is in the open position, the opening force results in friction on each link so that they are in the locked pivot position.

A shape-memory alloy is an alloy that can be deformed when cold but returns to its pre-deformed (“remembered”) shape when heated. Shape-memory alloys are copper-aluminium-nickel and nickel-titanium (NiTi), but SMAs can also be created by alloying zinc, copper, gold and iron. Iron-based and copper-based SMAs, such as Fe—Mn—Si, Cu—Zn—Al and Cu—Al—Ni, NiTi-based SMAs are preferable for most applications due to their stability and practicability and superior thermo-mechanic performance. SMAs can exist in two different phases, with three different crystal structures (i.e. twinned martensite, detwinned martensite and austenite) and six possible transformation. Shape-memory polymers (SMPs) are polymeric smart materials that have the ability to return from a deformed state (temporary shape) to their original (permanent) shape induced by an external stimulus (trigger), such as temperature change. Linear block copolymers are polyurethanes with ionic or mesogenic components made by prepolymer method. Other block copolymers with the shape-memory effect include block copolymer of polyethylene terephthalate (PET) and polyethyleneoxide (PEO), block copolymers containing polystyrene and poly(1,4-butadiene), and an ABA triblock copolymer made from poly(2-methyl-2-oxazoline) and polytetrahydrofuran. A linear, amorphous polynorbornene (Norsorex, developed by CdF Chemie/Nippon Zeon) or organic-inorganic hybrid polymers consisting of polynorbornene units that are partially substituted by polyhedral oligosilsesquioxane (POSS) also have shape-memory effect. Some thermoplastic polymers, are shape memory such as PEEK.

As shown in FIG. 1D, the first adjustable mechanism 140 and the second adjustable mechanism 150 comprise a plurality of links 200 interconnected by a plurality of pins 210, according to one embodiment. The plurality of links 200 is shown to be three for the first and second adjustable mechanism; however, the number of the plurality of links 200 may be adjusted according to the angle of surgery or type of surgery requiring retracted tissue. In one embodiment, the number of links for the first and second adjustable mechanism may be between about 1 and about 20, alternatively, between 2 about and about 10. The number of links for the first and second adjustable mechanism may be equal or unequal depending on the angle of surgery or the type or surgery requiring retracted tissue. Each link 200 permits a pivot angle between about 0 and about 90 degrees with respect to adjacent links 200, as shown in FIG. 1E. Alternatively, each link 200 permits a pivot angle between about 0 and about 130 degrees with respect to the first arm 120 and the second arm 130, according to one embodiment. The first adjustable mechanism 140 includes a first mating junction 220 on the distal end and the second adjustable mechanism includes a second mating junction 230 on the distal end; wherein the first mating junction 220 and the second mating junction 230 connects the first retractor 160 and the second retractor 170, respectively. In one embodiment, the first mating junction 220 is connected to the first retractor 160 by way of a first screw 240, and the second mating junction 230 is connected to the second retractor 170 by way of a second screw 250. The first screw 240 and the second screw 250 may include a retaining ring, according to one embodiment. When it is in the open position, the pivot axis of the links is initially 90 degrees from the pivot axis of the handle . . . which provides friction or taut force so they remain locked when in the open position. The pivot axis of the links is offset from the pivot axis of the handle to provide a friction force on the link pivot axis from further rotation when in the open position. In one embodiment, the link pivot axis is non-parallel to the handle pivot axis, as to lock the link pivot axis when in the open position for tissue retraction. During the handle operation, the forces produced are normal to the handle pivot joint so the adjustment mechanism will not be able to rotate. The adjustment mechanism has only one single degree of freedom which is the rotation of +/−90 degree around the link pivot axis defined by the pin. Each solid body in the space from a kinematic point of view has 6 degree of freedom and in embodiment, the link has left only one degree of freedom. All the other 5 degrees of freedom are locked and they are able to provide a strength higher than the resistance forces produced by the body tissue within the limits expressed by a force between about 2 lb and about 25 lb.

The first adjustable mechanism and the second adjustable mechanism are adjustable by the user by exerting a force F on the pivot angle. The force F necessary may be between about 0.2 lb or just below a low joint friction as to avoid any additional shear forces on the body tissue in case of residual adjustment during the use of the surgical retractor and at the same time to hold the first claw and second claw in place. The force F may increase to about 25 lb, depending on the region of the body where the surgical retractor is being used The first adjustable mechanism and the second adjustable mechanism after moving to the bent configuration, will remain in the bent configuration unless a force greater than 0.2 lb is exerted about the pivot pin. The reason for the higher force is so the multipurpose retractor remains in the bent configuration during tissue retraction and does not lose its bent configuration. In one embodiment, the first adjustable mechanism and the second adjustable mechanism include a locking mechanism to prevent the first adjustable mechanism and the second adjustable mechanism from moving from the unbent configuration.

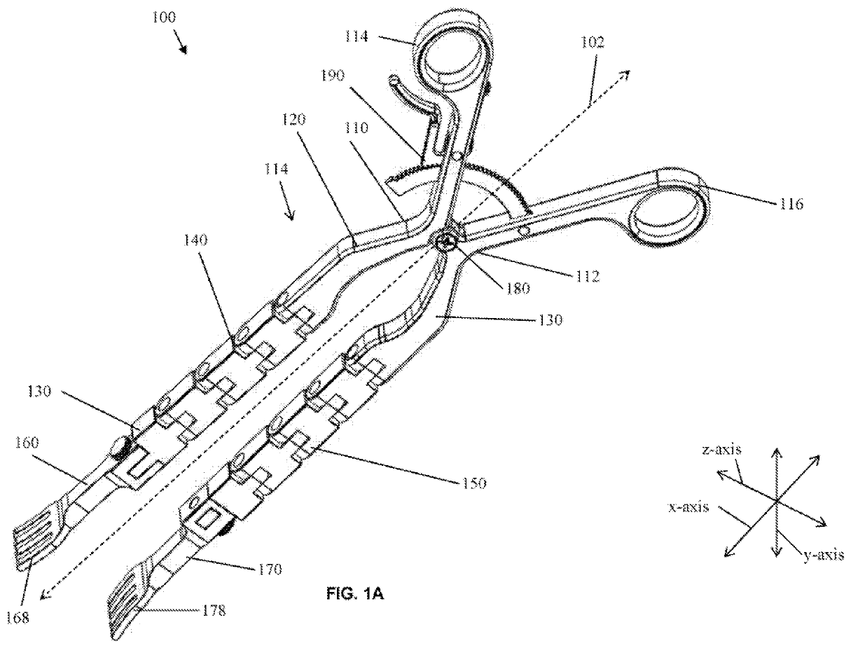

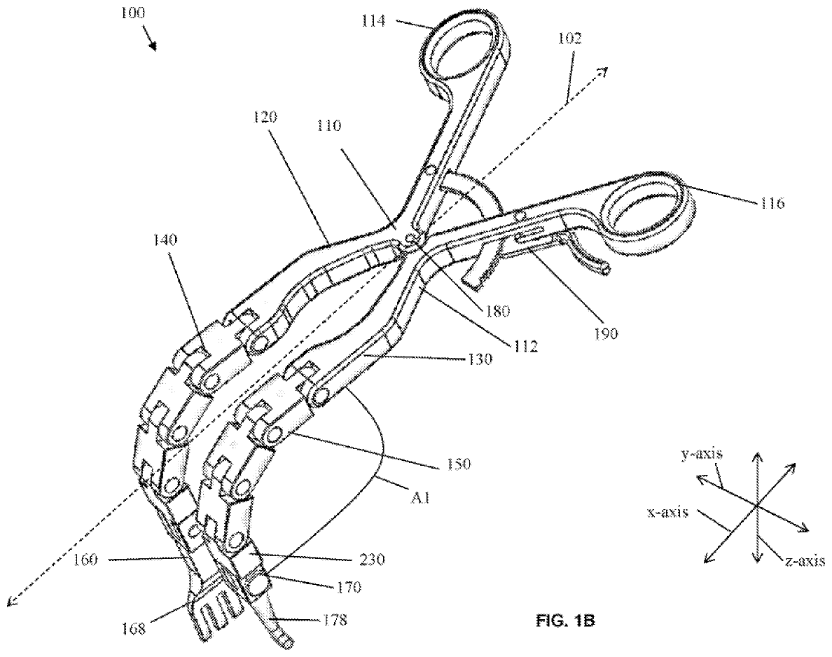

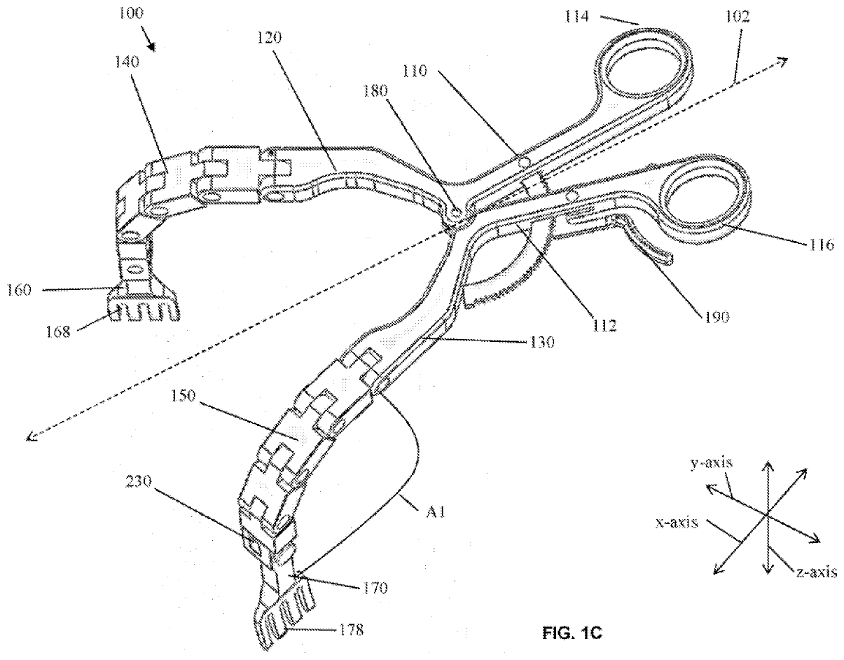

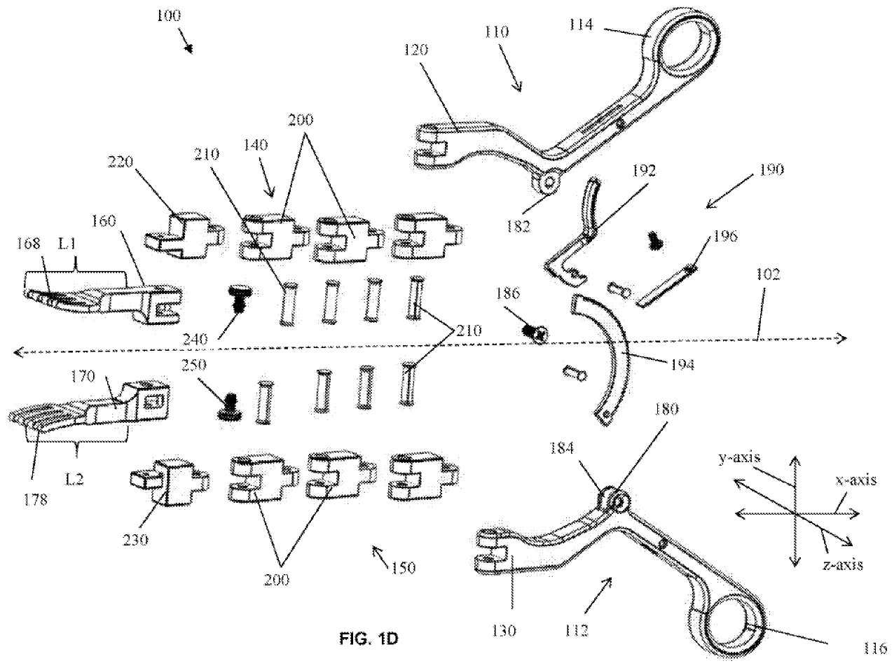

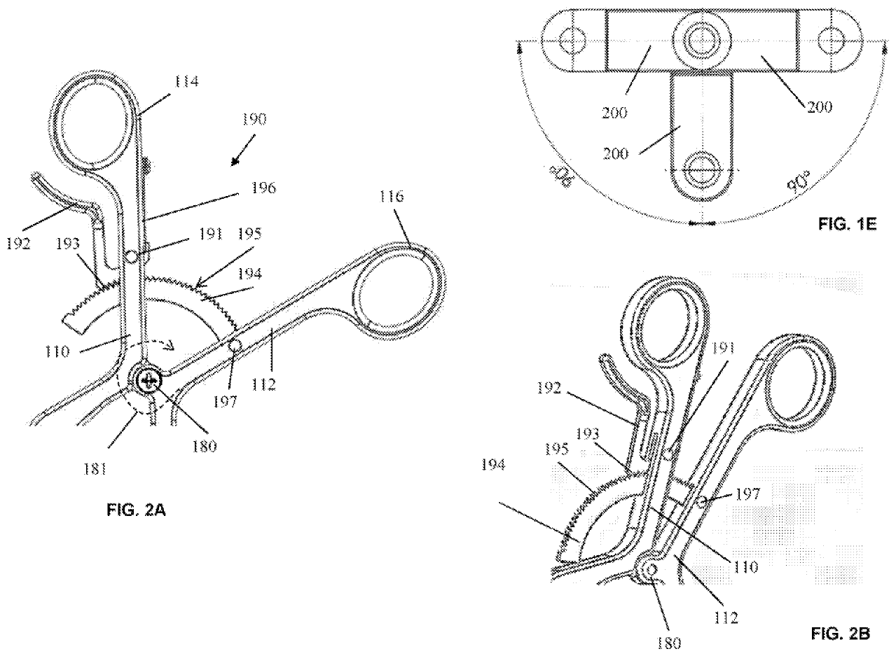

As shown in FIG. 1D, the first handle 110 includes a trigger mechanism 190 to keep the Multipurpose Retractor 100 in the open position and in the closed position, according to one embodiment. As shown in FIGS. 1D and 2A, the trigger mechanism 190 includes a trigger 192, a gear 194, and a spring line 196 operably coupled with the first handle 110 and the second handle 112. The gear 194 includes a generally curved configuration and is operably coupled with the first handle 110 and the second handle 112. The gear 194 is secured to the second handle 112 by way of a gear pin 197. The gear 194 includes a geared surface 195. The geared surface 195 operably locks with a geared surface 193 of the trigger 192 and prevents rotational movement of the first handle 110 and the second handle 112. The spring line 196 locks the trigger 192 into a secure position. When the trigger 192 is rotated about a trigger pin 191 disposed in the first handle 110, the geared surface 193 of the trigger 192 dislodges from the geared surface 195 of the gear 194, permitting rotational movement of the first handle 110 to rotate 181 about the pivot point 180 towards the second handle 112 and the gear 194 moves through the first handle 110 to the open position and retract tissue, as shown in FIG. 2B. In the open position, the trigger 192 is then biased to lock the geared surface 193 of the trigger 192 with the geared surface 195 of the gear 194 and prevent rotational movement of the first handle 110 and the second handle 112 as to keep tissue retracted. The first handle 110 includes a circular tab 182 in the middle portion of the first handle 110, and the second handle 112 includes a circular seat 184 in the middle portion of the second handle 112, as shown in FIG. 1D. The circular tab 182 sits in the circular seat 184 and is secured by a pivot screw 186 to permit rotational movement of the pivot point 180.

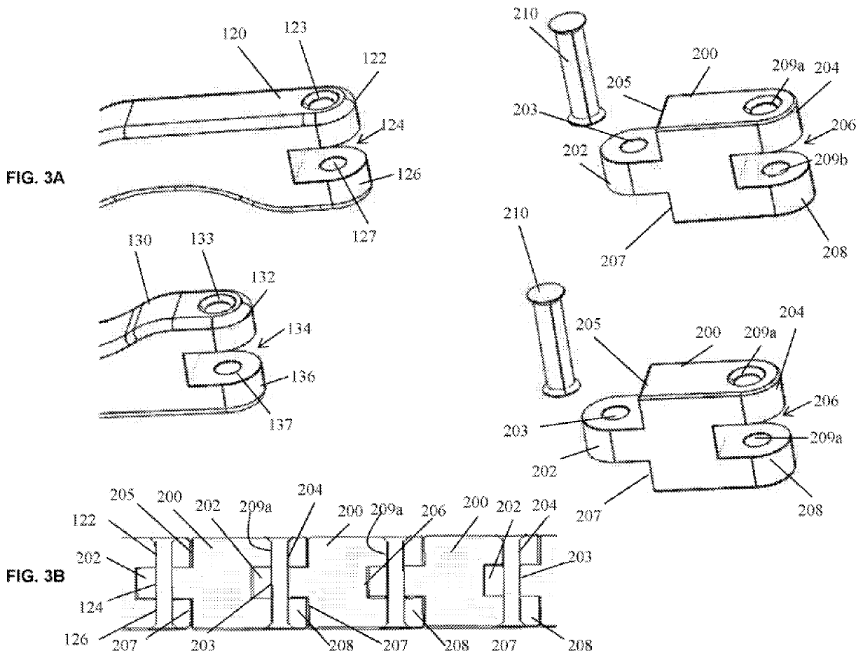

As shown in FIG. 3A, the first arm 120 includes a first distal top tab 122, a first top lumen 123, a first distal bottom tab 126 and a first bottom lumen 127. The second arm 130 includes a second distal top tab 132, a second top lumen 133, a second distal gap 134, a second distal bottom tab 136, and a second bottom lumen 137. In one embodiment, the first distal gap 124 is disposed between the first distal top tab 122 and the first distal bottom tab 126. In one embodiment, the second distal gap 134 is disposed between the second distal top tab 132 and the second distal bottom tab 136. The first distal top tab 122 and the first distal bottom tab include a generally circular outer surface to engage with the plurality of links 200. The second distal top tab 132 and the second distal bottom tab 136 include a generally circular outer surface to engage with the plurality of links 200. The generally circular outer surface permits the plurality of links 200 to rotate about the first arm 120 and the second arm 130. The plurality of links 200 are identical, according to one embodiment, and alternatively, the plurality of links 200 are different and include different lengths, according another embodiment. The plurality of links 200 include a proximal circular tab 202, a distal top tab 204, a distal gap 206, and distal bottom tab 208. The proximal circular tab 202 includes a proximal lumen 203 disposed there through and the pin 210 is coaxially disposed through the proximal lumen 203. The proximal circular tab 202 is operably disposed in the first distal gap 124 and includes a generally circular outer surface to permit the rotational movement of the link 200 about the first arm 120. The distal gap 206 is also operably coupled with the proximal circular tab 202 of another adjacent link 200. The distal top tab 204 includes a top lumen 209a disposed there through and the distal bottom tab 208 includes a bottom lumen 209b disposed there through. The pin 210 is coaxially disposed in the distal top tab 204 through the top lumen 209a, the proximal lumen 203 of the proximal circular tab 202, and the distal bottom tab 208 through the bottom lumen 209b. The distal top tab 204 and the distal bottom tab 208 include a generally circular outer surface to permit the rotational movement of the adjacent link 200. A proximal top surface 205 and a proximal bottom surface 207 abut the generally circular outer surface of the distal top tab 204 and the distal bottom tab 208 permitting the rotational movement. In one embodiment, the proximal circular tab 202 is between the proximal top surface 205 and the proximal bottom surface 207, as shown in FIG. 3B.

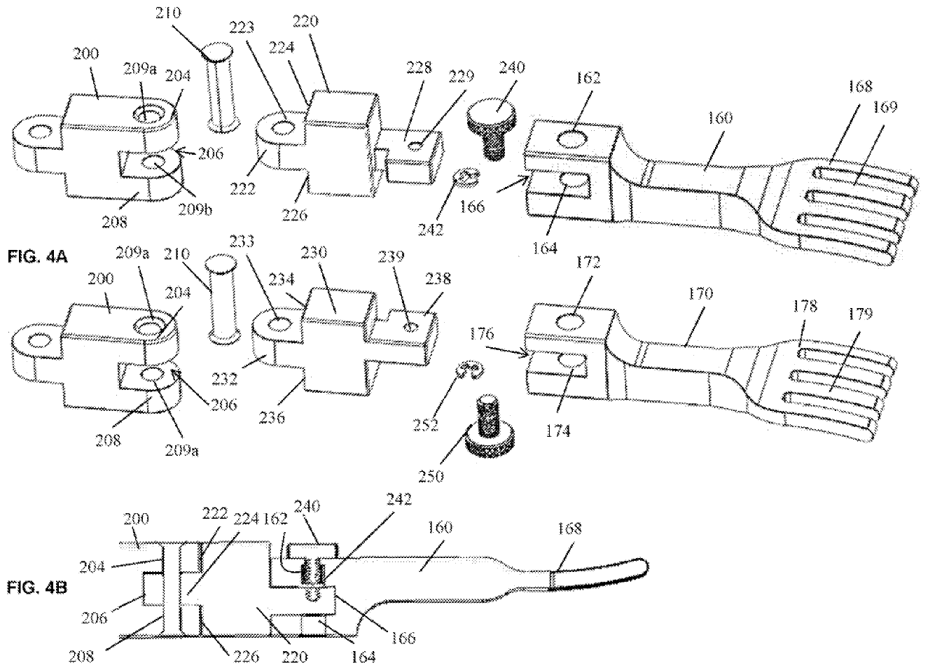

As shown in FIGS. 4A-4B, the plurality of links 200 is operably coupled with the first mating junction 220 and the second mating junction 230. The first mating junction 220 includes a first proximal tab 222, a proximal top surface 224, a proximal bottom surface 226, a middle section 227, and a first distal tab 228. The first proximal tab 222 includes a proximal lumen 223 and the first distal tab 228 includes a distal lumen 229. The first proximal tab 222 includes a generally circular outer surface that operably couples with the distal gap 206 of the link 200, and the proximal lumen 223 coaxially aligns with the distal top tab 204 and the distal bottom tab 208, whereby the pin 210 is coaxially disposed through the proximal lumen 223, the distal top tab 204, and the distal bottom tab 208. The generally outer surface of the distal top tab 204 and the distal bottom tab 208 abut against the proximal top surface 224 and the proximal bottom surface 226, respectively, permitting the first mating junction 220 to rotate about the pin 210.

The second mating junction 230 includes a second proximal tab 232, a proximal top surface 234, a proximal bottom surface 236, a middle section, and a second distal tab 238. The first proximal tab 222 includes a proximal lumen 233 and the second distal tab 238 includes a distal lumen 229. The second proximal tab 232 includes a generally circular outer surface that operably couples with the distal gap 206 of the link 200, and the proximal lumen 233 coaxially aligns with the distal top tab 204 and the distal bottom tab 208, whereby the pin 210 is coaxially disposed through the proximal lumen 233, the distal top tab 204, and the distal bottom tab 208. The generally outer surface of the distal top tab 204 and the distal bottom tab 208 abut against the proximal top surface 234 and the proximal bottom surface 236, respectively, permitting the second mating junction 230 to rotate about the pin 210. The first distal tab 228 and the second distal tab 238 operably couple with the first retractor 160 and the second retractor 170.

As shown in FIGS. 4A-4B, the first retractor 160 includes a proximal top opening 162, a proximal bottom opening 164, a proximal middle gap 166, a middle portion, and the first distal claw 168. The first retractor 160 is operably coupled with the first mating junction 220 by coaxially aligning the proximal top opening 162 and the proximal bottom opening 164 with the distal lumen 229 of the first distal tab 228 and coaxially disposing the first screw 240 there through. A first retaining ring 242 disposed on top of the first distal tab 228 to further secure the first screw 240 and the first mating junction 220 and the first retractor 160 together. The first distal claw 168 includes a plurality of claws 169 to secure and retract tissue. In one embodiment, the plurality of claws 169 may number between about 2 and about 10. The number of claws 169 may be adjusted for the type of tissue being retracted or the type of surgery.

The second retractor 170 includes a proximal top opening 172, a proximal bottom opening 174, a proximal middle gap 176, a middle portion, and the second distal claw 178. The second retractor 170 is operably coupled with the second mating junction 230 by coaxially aligning the proximal top opening 172 and the proximal bottom opening 174 with the distal lumen 239 of the first distal tab 228 and coaxially disposing the second screw 250 there through. A second retaining ring 252 disposed on top of the proximal bottom opening 174 to further secure the first screw 240 and the first mating junction 220 and the first retractor 160 together. The second distal claw 178 includes a plurality of claws 179 to secure and retract tissue. In one embodiment, the plurality of claws 179 may number between about 2 and about 10 and may equal the number of claws 169 on the first retractor 160 and may be unequal to the first retractor 160. The number of claws 169 may be adjusted for the type of tissue being retracted or the type of surgery.

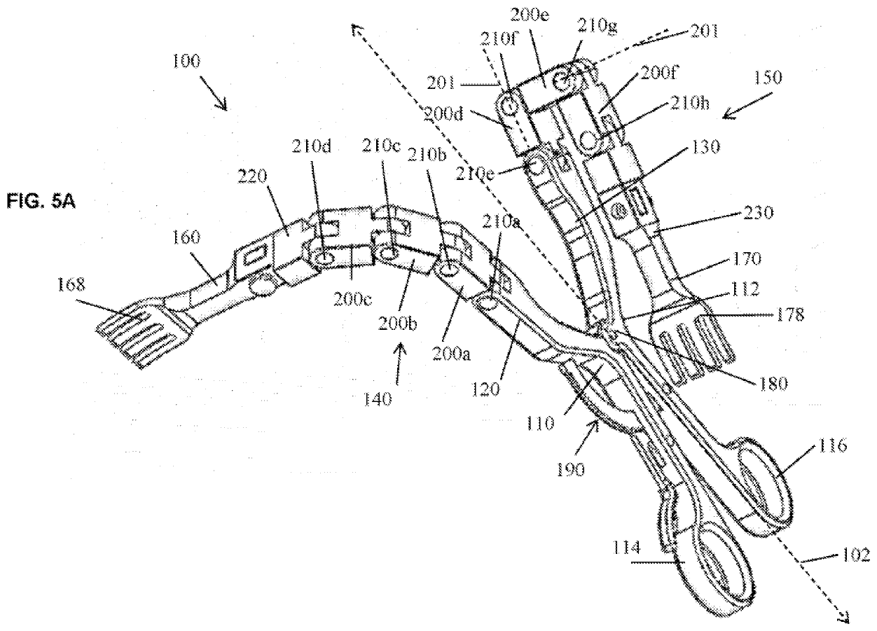

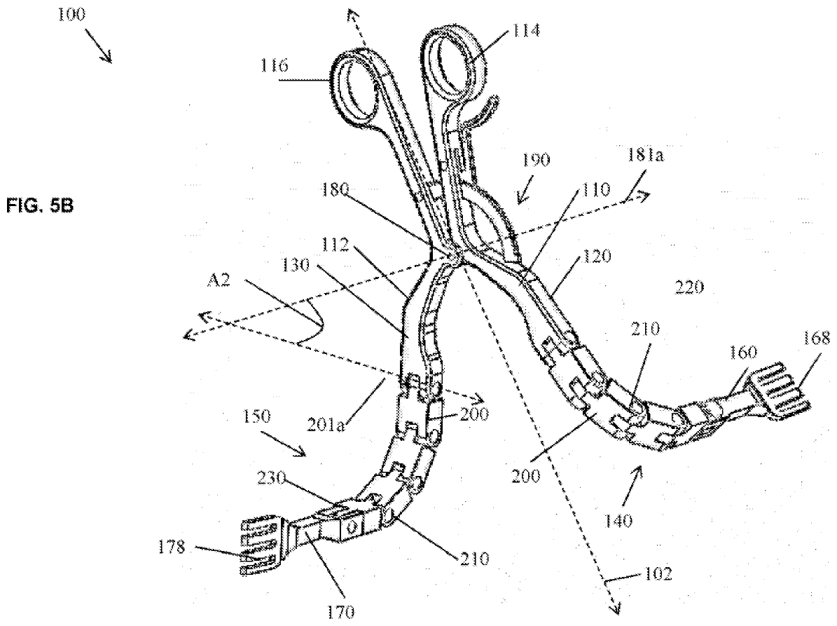

As shown in FIG. 5A, the multipurpose retractor 100 and the first adjustable mechanism 140 and the second adjustable mechanism 150 allow the retractors to be bent along a variety of angles. The multipurpose retractor 100 includes a first link 200a, a first pin 210a, a second link 200b, a second pin 210b, a third link 200c, a third pin 210c, a fourth link 200d, a fourth pin 210d, a fifth link 200e, a fifth pin 210e, a sixth link 200f, a sixth pin 210f, a seventh link 200g, a seventh pin 210g, and an eighth pin 210h, according to one embodiment. The first link 200a is coaxially coupled with the first arm 120 through first pin 210a. The second link 200b is coaxially coupled with the first link 200a through second pin 210b. The third link 200c is coaxially coupled with the second link 200b through third pin 210c. The first mating junction 220 is coaxially coupled with the third link 200c through fourth pin 210d. The fourth link 200d is coaxially coupled with the second arm 130 through fifth pin 210e. The fifth link 200e is coaxially coupled with the fourth link 200d through the sixth pin 210f. The sixth link 200f is coaxially coupled with the fifth link 200e through the seventh pin 210g. The second mating junction 230 is coaxially coupled with the sixth link 200f through the eighth pin 210h. Each link 200 includes a longitudinal axis 201 and is able to pivot about the longitudinal axis +/−90 degrees from the longitudinal axis 201. The fifth link 200e rotates about the longitudinal axis 201 about 90 degrees and the sixth link 200f rotates about the longitudinal axis 201, which allows the multipurpose retractor 100 to retract tissue about an 180 degree axis. The second adjustable mechanism bent about 180 degrees from the original longitudinal position. The multipurpose retractor 100 can retract tissue +/−about 180 degrees from the longitudinal axis 102. In one embodiment, one link 200 may be used with the multipurpose retractor if the rotation about the longitudinal axis requires at least about 90 degree rotation. As shown in FIG. 5B, the multipurpose retractor 100 shows that the first adjustable mechanism 140 and the second adjustable mechanism can be bent in opposite directions from the longitudinal axis 102. The pivot point 180 includes a pivot axis 181a that is generally offset and at angle A2 from the pivot axis 201a from the links 200. When the multipurpose retractor 100 is in the closed position, the pivot axis 201a of the links is initially about 90 degrees offset from the pivot axis 181a of the handle. The angle A2 provides friction or taut force so the links 200 remain locked and do not pivot when in the open position, allowing for proper retraction of tissue. The pivot axis 201a of the links is offset from the pivot axis 181a of the handle to provide a friction force on the link pivot axis from further rotation when in the open position. The offset angle is selected to be between about 5 degrees to about 90 degrees, according to one embodiment.

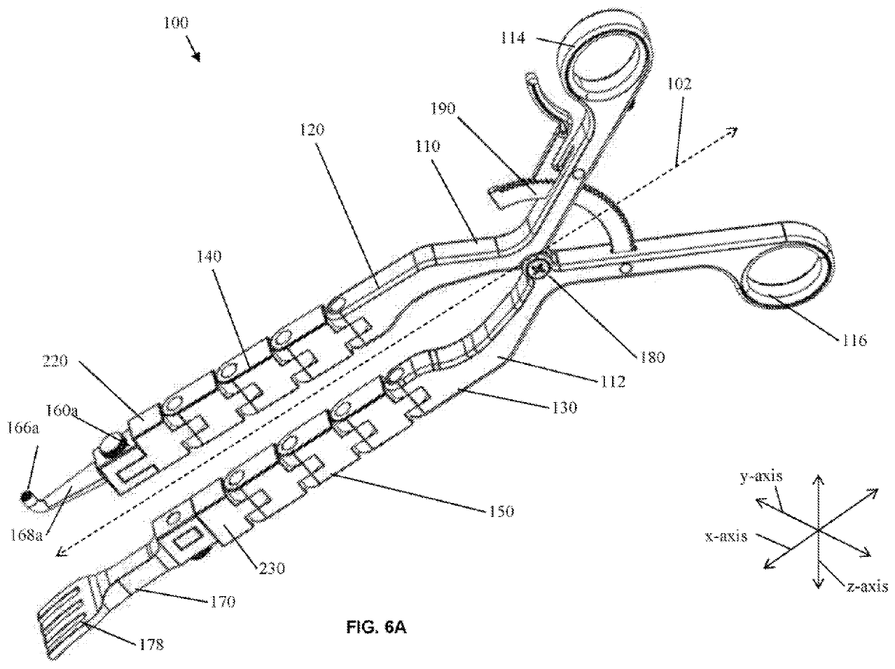

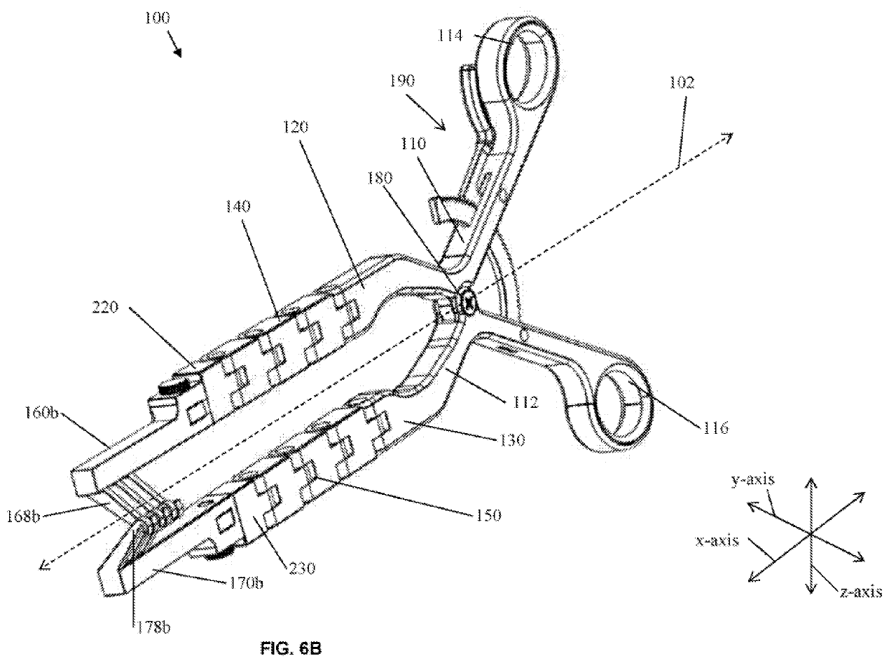

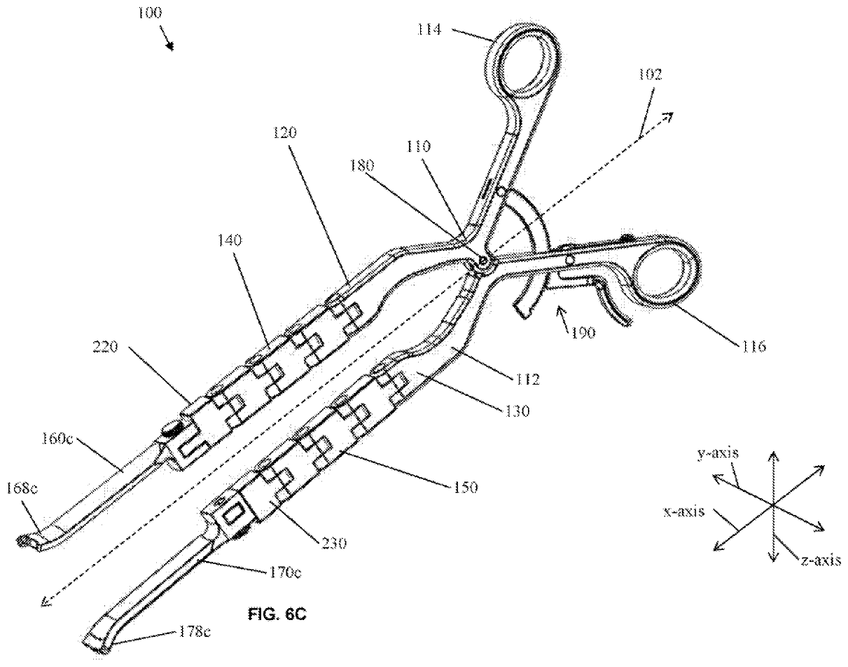

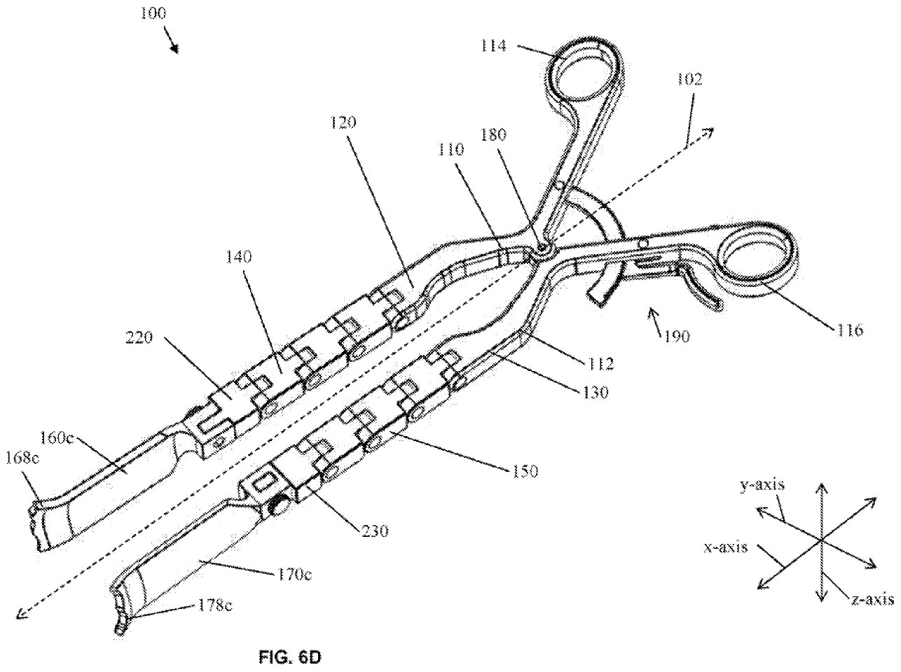

Different types of blades can be connected and disconnected to the handle via a mating junction as well as replacement of the blades if damaged or requiring sterile application. In one embodiment, the first retractor 160a includes a first distal hook 168a and the second retractor 170 includes a second distal claw 178, wherein the first distal claw 168 and the second distal claw 178 are operable to retract tissue when operable from the closed position to the open position, as shown in FIG. 6A. In another embodiment, the first retractor 160b includes a plurality of distal hooks 168b and the second retractor 170b includes a plurality of distal hooks 178b, as shown in FIG. 6B. In one embodiment, the plurality of distal hooks 168b and 178b is an angled rake. The plurality of distal hooks 168b and the plurality of distal hooks 178b interlock when in the closed position and unbent configuration. In another embodiment, the first retractor 160c includes a first flat blade 168c and the second retractor 170c includes a second flat blade 178c, as shown in FIG. 6C. The first flat blade 186c and the second flat blade 178c operate as a narrow cervical blade retraction including a width between about 10 mm and about 16 mm. The first flat blade 168c and the second flat blade 178c operate to opposing ends for narrow cervical procedures. In another embodiment, the first retractor 160d includes a first wide blade 168d and the second retractor 170d includes a second wide blade 178d, as shown in FIG. 6D. The first wide blade 168d and the second wide blade 178d operate to opposing ends for wide cervical procedures. The first and second wide blade is about 50% wider than the first and second narrow blade for wide cervical blade retraction and includes a width between about 20 mm and about 28 mm, according to one embodiment. As shown in FIGS. 6A-6D, others embodiment of the Multipurpose Retractor 100 comprises a first handle 110 and a second handle 112, wherein the first handle 110 includes a first arm 120 and the second handle 112 includes a second arm 130 extending to a distal end. A first adjustable mechanism 140 extends from the first arm 120 and a second adjustable mechanism extends from the second arm 130, wherein the first adjustable mechanism and the second adjustable mechanism pivot about a longitudinal axis 102 by angle A1. A first retractor 160a extends from the first adjustable mechanism 140 and a second retractor 170 extending from the second adjustable mechanism 150, wherein the first retractor 160 and second retractor 170 are operable to retract tissue when the Multipurpose Retractor moves to the open position in the bent configuration, as shown in FIG. 1C. The first arm 120 and the second arm 130 are operable in a closed position and an open position through a pivot point 180 disposed between the first arm and a first handle and the pivot point disposed between the second arm and a second handle.

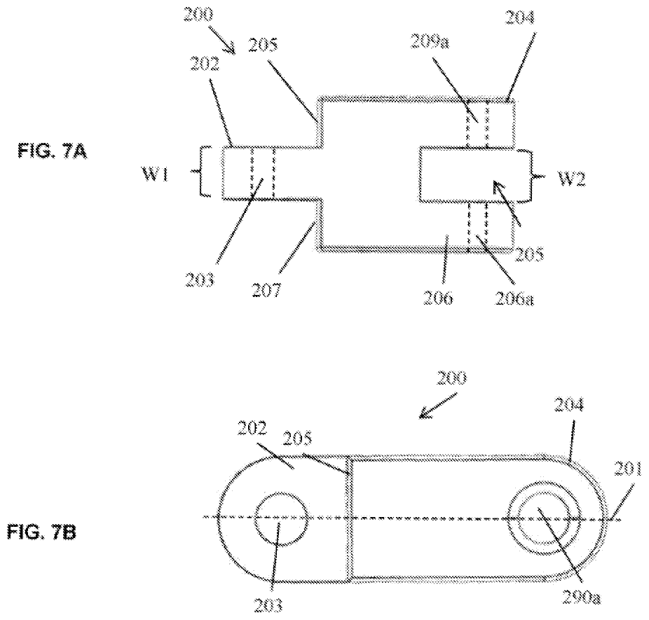

As shown in FIG. 7A, the link 200 is shown with the proximal circular tab 202 of including a width W1. The distal gap 206 includes a width W2, wherein the width W1 of the proximal circular tab 202 fits within the width W2 of the distal gap 206 to permit the proximal circular tab 202 rotate between the distal top tab 204 and the distal bottom tab 208. The top lumen 209a traverses the thickness of the distal top tab 204, the proximal lumen 203 traverses the thickness of the proximal circular tab 202, and the bottom lumen 209b traverses the thickness of the distal bottom tab 208. The distal top tab 204 and the distal bottom tab 208 include a generally circular outer surface to permit the rotational movement of the adjacent link 200, as shown in FIG. 7B. Each link 200 can rotate about the longitudinal axis 201 through the top lumen 209a and the proximal lumen 203. The proximal top surface 205 and a proximal bottom surface 207 include a generally flat surface to permit the distal top tab 204 and the distal bottom tab 208 for the rotational movement about the proximal lumen 203 when coaxially coupled with the pin 210. In one embodiment, the top lumen 209a includes a lip portion to permit coupling of the pin 210 and permit rotation movement.

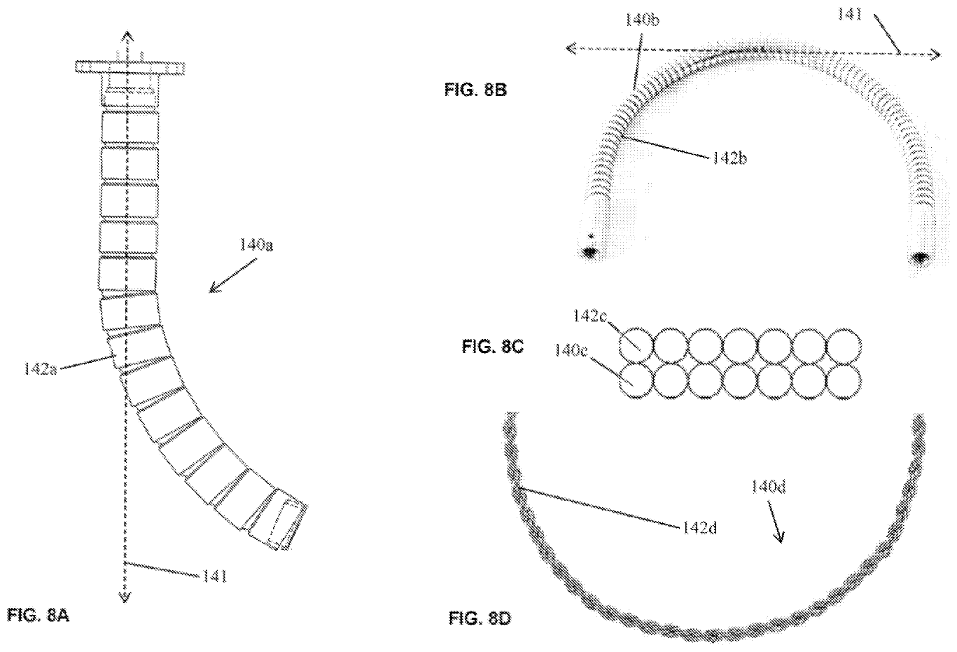

Alternative first adjustable mechanism and second adjustable mechanisms are shown in FIGS. 8A-8D. The first adjustable mechanism and the second adjustable mechanism may be identical, as shown previously, or alternatively, the first adjustable mechanism and the second adjustable mechanism may be different and selected from the following in any particular combination. As shown in FIG. 8A, the adjustable mechanism 140a includes a plurality of cylindrical links 142a that are bendable along the longitudinal axis 141, the lateral axis, and the vertical axis. As shown in FIG. 8B, the adjustable mechanism 140c is a metal cable 142b is bendable along the longitudinal axis 141, the lateral axis, and the vertical axis. As shown in FIG. 8C, the adjustable mechanism 140c is a plurality of interconnected spheres 142c that is bendable along the longitudinal axis, the lateral axis, and the vertical axis. As shown in FIG. 8D, the adjustable mechanism 140d is a braided polymer 142d that is bendable along the longitudinal axis, the lateral axis, and the vertical axis.

A method of using a multipurpose surgical retractor, comprising the steps: disposing a handle including a distal end and a proximal end, extending a first arm and a second arm from the distal end; extending a first adjustable mechanism from the first arm and extending a second adjustable mechanism from the second arm, rotating the first adjustable mechanism and the second adjustable about a longitudinal axis and a lateral axis; and extending a first retractor from the first adjustable mechanism and extending a second retractor from the second adjustable mechanism, operating the first arm and the second arm from a closed position to an open position through a pivot point to retract tissue with the first retractor and the second retractor when in the closed position. The method further comprises interconnecting the first adjustable mechanism and the second adjustable mechanism with a plurality of links and a plurality of pivot links. The method further comprises, disposing the pivot point between the first arm and a first handle and disposing the pivot point between the second arm and a second handle. The method further comprises including a first distal claw extending from the first retractor and including a second distal claw extending from the second retractor; and retracting tissue with the first distal claw and the second distal claw when in the closed position. The method further comprises including a length L1 for first distal claw and including a length L2 for the second distal claw, wherein L1 and L2 are equal and a length to retract tissue. The method further comprises including a first gripping portion on the proximal end of the first handle and including a second gripping portion on the proximal end of the second handle; and translating the first gripping portion and the second gripping portion to pivot of the first arm and the second about the pivot point from the closed position to the open position. The method further comprises the first adjustable mechanism and the second adjustable mechanism is selected from the group consisting of: interconnected links, chains, braided polymers, cylinders, linked spheres, and metal cables. In one embodiment, adjustability can be accomplished via the use of malleable materials such as memory metals and polymers.

The method further comprises including a first mating junction on the distal end of the first adjustable mechanism and a second mating junction on the distal end of the second adjustable mechanism; and replacing the first retractor and the second retractor by way of the first mating junction and the second mating junction. The method further comprises operating the multipurpose surgical retractor for spinal orthopedics, ENT, neurosurgical, cardiac or podiatry operations.

All publications and patent applications mentioned in this specification are herein incorporated by reference to the same extent as if each individual publication or patent application was specifically and individually indicated to be incorporated by reference. While the invention has been described in connection with various embodiments, it will be understood that the invention is capable of further modifications. This application is intended to cover any variations, uses or adaptations of the invention following, in general, the principles of the invention, and including such departures from the present disclosure as, within the known and customary practice within the art to which the invention pertains.

What is claimed is:

1. An multipurpose surgical retractor comprising:
a handle comprising a distal end and a proximal end, wherein the proximal end includes a first arm and a second arm extending from the distal end;
a first adjustable mechanism extending from the first arm and a second adjustable mechanism extending from the second arm, wherein the first adjustable mechanism and the second adjustable mechanism are rotatable about a longitudinal axis and a lateral axis; and
a first retractor extending from the first adjustable mechanism and a second retractor extending from the second adjustable mechanism, wherein the first retractor and the second retractor are operable to retract tissue.

2. The multipurpose surgical retractor of claim 1, wherein the first arm and the second arm are operable in a closed position and an open position through a pivot point disposed between the first arm and a first handle and the pivot point disposed between the second arm and a second handle.

3. The multipurpose surgical retractor of claim 2, wherein the first adjustable mechanism and the second adjustable mechanism are selected from the group consisting of: interconnected links, chains, linked spheres, braided polymers, cylinders, and metal cables.

4. The multipurpose surgical retractor of claim 3, wherein the first retractor includes a first distal claw and the second retractor includes a second distal claw; and the first distal claw and the second distal claw are operable to retract tissue when operable from the closed position to the open position.

5. The multipurpose surgical retractor of claim 4, wherein the first distal claw includes a length L1 and the second distal claw includes a length L2, wherein L1 and L2 are equal and a length to retract tissue.

6. The multipurpose surgical retractor of claim 5, wherein the first handle includes a first gripping portion on the proximal end and the second handle includes a second gripping portion on the proximal end; and the first gripping portion and the second gripping portion assist in pivoting the first arm and the second arm about the pivot point from the closed position to the open position.

7. The multipurpose surgical retractor of claim 2, wherein the first adjustable mechanism and the second adjustable mechanism comprise a plurality of links interconnected by a plurality of pivot links.

8. The multipurpose surgical retractor of claim 6, wherein the first adjustable mechanism includes a first mating junction on the distal end and the second adjustable mechanism includes a second mating junction on the distal end; wherein the first mating junction and the second mating junction permits replacement of the first retractor and the second retractor.

9. The multipurpose surgical retractor of claim 6, wherein the first mating junction of the first adjustable mechanism is operably disposed in an opposite direction of the second mating junction of the second adjustable mechanism.

10. A method of using a multipurpose surgical retractor, comprising:
disposing a handle including a distal end and a proximal end, extending a first arm and a second arm from the distal end;
extending a first adjustable mechanism from the first arm and extending a second adjustable mechanism from the second arm, rotating the first adjustable mechanism and the second adjustable mechanism about a longitudinal axis and a lateral axis; and
extending a first retractor from the first adjustable mechanism and extending a second retractor from the second adjustable mechanism, operating the first arm and the second arm from a closed position to an open position through a pivot point to retract tissue with the first retractor and the second retractor when in the closed position.

11. The method of claim 10, further comprising interconnecting the first adjustable mechanism and the second adjustable mechanism with a plurality of links and a plurality of pivot links.

12. The method of claim 11, disposing the pivot point between the first arm and a first handle and disposing the pivot point between the second arm and a second handle.

13. The method claim 12, including a first distal claw extending from the first retractor and including a second distal claw extending from the second retractor; and retracting tissue with the first distal claw and the second distal claw when in the closed position.

14. The method of claim 13, including a length L1 for the first distal claw and including a length L2 for the second distal claw, wherein L1 and L2 are equal and a length to retract tissue.

15. The method of claim 14, including a first gripping portion on the proximal end of the first handle and including a second gripping portion on the proximal end of the second handle; and translating the first gripping portion and the second gripping portion to pivot of the first arm and the second adjustable mechanism about the pivot point from the closed position to the open position.

16. The method of claim 10, wherein the first adjustable mechanism and the second adjustable mechanism are selected from the group consisting of: memory metals, fluid hydraulics, interconnected links, chains, linked spheres, braided polymers, cylinders, and metal cables; and where the interconnected links are fixed in position selected from the group consisting of frictional pivots, memory metal, fluid hydraulics, and temperature sensitive materials.

17. The method of claim 16, further comprising including a first mating junction on the distal end of the first adjustable mechanism and a second mating junction on the distal end of the second adjustable mechanism; and replacing the first retractor and the second retractor by way of the first mating junction and the second mating junction.

18. The method of claim 16, further comprising operating the multipurpose surgical retractor for a surgical procedure selected from the group consisting of: spinal surgery, general orthopedics, trauma surgery, general surgery, plastic surgery, spinal orthopedics, Ear Nose and Throat (ENT) procedures, neurosurgical, cardiac procedures, veterinary surgery, and other surgical operations.

19. The method of claim 16, further comprising operably disposing the first mating junction of the distal arm in the opposite direction of the second mating junction of the second arm, and using similarly directed retractors are used in the first mating junction and the second mating junction.
